(19) United States
(12) Patent Application Publication
(10) Pub. No.: US 2003/0100739 A1
(43) Pub. Date: May 29, 2003

(54) Method for producing cross-linked hyaluronic acid-protein bio-composites

(75) Inventors: Shiao-Wen Tsai, KaoHsiung (TW); Jui-Hsiang Chen, Hsinchu (TW); Chiung-Lin Yang, Taipei (TW)

Correspondence Address: DOUGHERTY & TROXELL, SUITE 1404, 5205 LEESBURG PIKE, FALLS CHURCH, VA, 22041, US

(73) Assignee: INDUSTRIAL TECHNOLOGY RESEARCH INSTITUTE

(21) Appl. No.: 10/076,288

(22) Filed: Feb. 19, 2002

(30) Foreign Application Priority Data
Aug. 10, 2001 (TW) 90119567

(51) Int. Cl. A01N 37/18; A61K 38/00; C07K 1/00; C07K 14/00; C07K 17/00; C09H 3/00; C09H 3/02; A61K 38/17

(52) U.S. Cl. 530/354; 514/2

(57) ABSTRACT

This invention is concerned with a new method for producing different types of cross-linked hyaluronic acid—protein bio-composites. In the process of manufacture, polysaccharide and protein solution are mixed under moderate pH values and salts, and the well mixed solution can be prepared to various types, such as membrane, sponge, fiber, tube or micro-granular and so on. After then, with the bio-compatibility, biodegradation, prolong enzymatic degradation and good physical of bio-composite is formed in the little acid of organic solution that with the cross-linked reagent.

### BACKGROUND OF THE INVENTION

1. Field of Invention:

This invention relates generally to a new method for producing different types of cross-linked hyaluronic acid—protein bio-composites, and in particular, to a homogenous solution that formed by various rates of hyaluronic acid—protein, and can be processed to different types of the bio-composites.

2. Description of the Related Art

Hyaluronic acid (HA) is a muco- polysaccharide occurring naturally in the vertebrate tissues and fluid, and having a linear high molecular weight usually varying within the range of several thousands to several millions daltons depending on its source and purification method. HA was first reported by Karl Meyer et al. in 1934, HA contains glucuronic acid and glucosamine which was isolated and purified from the vitreous humor of cow. HA consists of alternating N-acetyl-D-glucosamine and D-glucuronic acid residues joined by alternating, beta 1-3 glucuronic and beta 1-4 glucosaminidic bonds, so that the repeating unit (dimer) is (1 fwdarw.4)-beta-D-GlcA- (₁.fwdarw.3)-beta-D-GlcNAc-. HA is widely distributed in connective tissues, mucous tissue, crystalline lens and capsules of some bacteria. In commercial applicability, HA has been as a matrix in drug delivery, arthritic agent, arthritic operation or wound healing. In industrial production, HA was mainly extracted and purified from the cockscomb, but HA can also be isolated from the capsules of Streptococci that produced in a ferment by bio-technique.

HA solution shows a high viscousness and flexibility. The characteristics of HA that applied in the ophthalmology is named as a viscoelastic matrix. These characteristics were produced due to the formation of polymeric network by the high MW and molecular volume of HA. HA is synthesized by the HA synthetase that exists in the plasma membrane, and hydrolyzed by the hyaluronidase that exists in lysozyme. The interaction of HA and proteoglycans can stabilize the structure of matrix and modify the behavior of cell surface. This characteristic provides many important physiological functions, including: lubrication, water-sorption, water retention, filtration, and modulates the distribution of cytoplasmic protein.

It has been reported that HA has with many functions of (1) nationally occurring in body, (2) non immune reaction, (3) degradation and absorption by human body, (4) mass production, (5) application in the high bio-molecular of medicine. The major application of HA is the ophthalmic operation of cataract and cornea damage. High molecular of HA solution is injected into the eye as a viscoelastic fluid, and plays a special role to maintain the morphology and function of eye. HA has been recently applied in wound healing, anti-adhesion and drug delivery. HA is present between cells as a complex with protein in tissue, forms a jelly matrix owing to it high water retention and can be useful for comestible and play an important role in anti-skin aging.

Collagen is a structure protein found in animals. It is a naturally bio-molecular, and can eliminate the immune-reaction via isolation, purification or treatment with enzyme (such as pepsin), and get a good bio-compatibility of collagen. Collagen can via various reconstruction, chemical cross-linking technique and processing procedure different types, such as plate, tube, sponge, powder or soft fiber. Collagen is a biodegradable and low toxic polymer in the body. It has been used as a hemostatic agent, nerve regeneration, tissue anaplastic, scald dressing, hernia repair, urethra operation, drug delivery, ophthalmology, vaginal contraceptive, cardiac valve repair, blood vessel operation and operating structure, and other biomedical materials.

Gelatin is a denatured collagen. The amino acid content is similar to the collagen but structure and chemic-physical properties are different. Up to date, it has been used in a wide variety of food application and medical research, such as hemostatic cotton and drug delivery.

HA and collagen are the major contents of extra-cellular matrix. Gelatin is also made from collagen. Therefore, gelatin is also with good bio-compatibility and biodegradation in the body. The gelatin composites can be also used for the development of the implant matrices in the biomedical materials, such as histological engineering, release system of material or anti-adhesive materials.

(1 ) Milena Rehakova et al., 1996,Journal of biomedical materials research, vol. 30,page 369-372,describes the method for preparing collagen and hyaluronic acid composite materials with the glyoxal and starch dialdehyde as a cross-linker. The collagen was dispersed in 0.5 M acetic acid solution, and then HA was added to the solution for 5 mins. A fiber precipitate was formed and filtered, washed several times with water and alcohol, and dried at a temperature of 35° C., and then a smooth surface of fiber structure was produced. The cross-linking of composite material was carrier out in starch dialdehyde solution, but the cross-linking of glyoxal was carrier out when HA and glyoxal were added to the suspension of collagen, or added glyoxal to the suspension of collagen first and then added HA.

(2 ) Jin-Wen Kuo et al., 1991,Bio-conjugate chemistry,vol,2,page 232-241,describes a method for preparing water-insoluble derivatives of hyaluronic acid by reacting high molecular HA with the 1-ethyl-3-(3-dimethylaminopropyl) carbodiimide at a pH of 4.75. In a general experiment, sodium hyaluronate was dissolved in distilled water to produce a 4 mg/ml HA solution. In some reaction, the amine and sodium hyaluronate were mixed together. The pH of the aqueous solution was adjusted to pH 4.75. Carbodiimide was dissolved in water or isopropanol, depended on the solubility.

After the mix of HA and carbodiimide, a pH of 4.75 was maintained by addition of 0.1 N HCl using a pH Stat apparatus. The reaction mixture was kept at room temperature for 2hrs, then 5% (weight/volume) reaction solution of HCl was added, and then a precipitate is formed after adding 3 time volume solution of ethanol. Non-reacted chemical reagent was washed out for 2-3 times with distilled water. Finally, the precipitate was dissolved in deionozed water before lyophilization.

(3 ) Lin-Shu Liu et al., 1999,Biomaterials, vol, 20,page 1097-1108,States a method for preparation of hyaluronate-polyaldehyde by treatment of hyaluronate with sodium periodate. Hyaluronate-polyaldehyde was prepared by oxidizing sodium hyaluronate with sodium periodate. A collagen-hyaluronate matrix was synthesized by the covalent biding of aldehyde group to the collagen, and can be provide to support the growth of cartilage tissue or bone repair material.

(4 ) D. Bakos et al., 1999,Biomaterials, vol, 20,page 191-195,describes a new method for preparing the composite bio-material. The composite material consisted of nine parts of inorganic components by weight and one part of organic component, including 92 wt % collagen and 8 wt % hyaluronic acid. The fraction of hydroxapatite particles was gradually added to the solution of hyaluronic acid in de-ionized water, and intensively mixed. Separately, the dispersion of very fine collagen fibers (1% by dry weight )in de-ionized water was prepared after dry fibrillation of lyophilized fibers of collagen. The two prepared dispersions were mixed together to form the complex precipitate. The precipitate was filtered and dried at a temperature of 37° C. in PTFE form.

(5) C. J. Doillon et al., 1988,Biomaterials, uses a porous sponge of collagen to support the growth of epithelium and fibroblast cell, and as a matrix of artificial skin. HA and/or fibronectin can enhance the repair of skin wound and the proliferation of cell. These high molecular can modify the behavior of tissue culture. The method of preparation was that the water-insoluble collagen (1% by weight) was dispersed in hydrogen chloride solution at a pH 3.0. In this step, 1% w/w of hyaluronic acid, fibronectin, dermatan sulfate and chondroitin-6-sulfate were added to collagen solution. The dispersion solution was frozen at −30° C., and then lyophilized before cross-linking.

(6 ) S. Srivastava et al., 1990,Biomaterials, vol, 11, page 155-161,indicates that added the glucosaminoglycans, (5% or 10% chondroitin sulfate and less 5% HA ) on the collagen gels will enhance the cell growth and adhesion, the growth of cells but more 5% HA incorporated into collagen gels inhibited cell adhesion and growth.

(7 ) S. Srivastava et al., 1990,Biomaterials, vol, 11, page 162-168,estimated the effect of the collagen or modified collagen on the growth of fibroblast cell line. The preparation of collagen/GAGs and fibronectin composite materials were following as Yannas described. The 3% w/v of degassed collagen slurry was stirred in 0.05 M acetic acid solution. The solution of HA that dissolved in 0.05 M acetic acid was added to the solution until the dry weight of collagen was 2.5%, and then solution was homogenized and degassed. Collagen/HA composite material comprised 5%, 10%, 20% GAGs, and collagen/CS composite material comprised 5%, 10% chondroitin-4-sulfate and chondroitin-6-sulfate. The method of preparing was same as the above described. The 1% fibronectin was added to the above composite material, and placed on the petri dish for cell culture. Experimental results showed that polystyrene was better than nature collagen to be a material of petri dish, but the adhesion of nature collagen was improve by chemical modification or added the fibronectin and chondroitin-4-sulfate. As the content of HA was more 5%, however, the cell adhesion and growth of nature collagen matrix could be better than the polystyrene material.

(8 ) M. Hanthamrongwit et al., 1996,Biomaterials, vol, 17, page 775-780,studies the effect of the glycosaminoglycans, hyaluronic acid and chondroitin-6-sulfate, diamines and a carbodiimide cross-linking agent on the growth of human epidermal cells on collagen gels. Collagen gel (0.3% w/v) was formed by mixing 4.2 mg/ml collagen solution, a mixture of 10 X DMEM and 0.4 M NaOH(2:1 )and 1:100(v/v)acetic acid at a ratio of 7:1:2 and adjusting the pH to 8-8.5 with 1 M NaOH. The gels were allowed to set completely for 2 hrs at room temperature. GAG solutions were prepared at 3 mg/ml in 1 X serum-free DMEM and incorporated into the collagen solution at various percentages by replacing the 2 volumes of acetic acid in the above ratio. 1-ethyl-3-(3-dimethylaminopropyl carbodiimide) and diamine incorporated in the gels was used as a cross-linking agent.

(9 ) L.H.H. Olde Damink et al., 1996,Biomaterials,vol,17,page 765-773,treats the cross-linking of non-cross-linked DSC (dermal sheep collagen) with EDC to give E-DSC was performed by immersing N-DSC samples weighting 1 g (1.2 mmol carboxylic acid groups) in 100 ml of an aqueous solution containing 1.15 g (6.0 mmol ) EDC at room temperature for 18 hrs. During the reaction, a pH of 5.5 was maintained by addition of 0.1 M HCl using a pH Stat apparatus. The molar amount of carboxylic acid group (—COOH) of N-DSC samples was calculated assuming that 120 carboxylic acid group containing residues are present per α-chain(1000 amino acids) and that each α-chain has a molecular weight of 100,000. After cross-linking, E-DSC samples were washed for 2 hrs in a 0.1 M Na₂HPO₄ solution and subsequently washed four times with distilled water before lyophilization. The other cross-linking of N-DSC with EDC and NHS to give E/N-DSC was performed by immersing N-DSC samples in aqueous solution containing EDC and NHS at room temperature for 4 hrs. The results showed that addition of N-hydroxylsuccinimide to the EDC-containing cross-linking solution (E/N-DSC ) increased the rate of cross-linking.

(10) Yannas et al., 1997, U.S. Pat. No. 4,060,081,states a multilayer membrane which is useful as synthetic skin. Preferred materials for the first layer are cross-linked composites of collagen and a muco-polysaccharide. A second layer is formed from a nontoxic material which controls the moisture flux of the overall membrane.

(11) Yannas et al., 1981, U.S. Pat. No. 4,280,954,states a method for preparing cross-linked collagen-muco-polysaccharide composite materials. A collagen solution at pH 3.2 and muco-polysaccharide solution (weight ratio is 6%-15% by weight) were mixed together, and then a precipitate of aldehyde covalent cross-linked collagen-muco-polysaccharide composite was formed.

(12) Yannas et al., 1982, U.S. Pat. No. 4,350,629 discover that if collagen fibrils in an aqueous acidic solution (<pH 6.0) are contacted with a cross-linking agent (glutaraldehyde) before being contacted with glycosaminoglycan, the materials produced have extremely low level of thrombogenicity. Such materials are well suited for in-dwelling catheters, blood vessel grafts, and other devices that are in continuous contact with blood for long periods of time.

(13 ) Yannas et al., 1984, U.S. Pat. No. 4,448,718,describes a process for preparing a cross-linked collagen- glycosaminoglycan composite material which comprises forming an uncross-linked composite material from collagen and a glycosaminoglycan and containing the uncross-linked composite with a gaseous aldehyde until a cross-linked product having an M. sub. C of from about 800 to about 60,000 is formed.

(14) Balazs et al., 1986, U.S. Pat. No. 4,582,865,states a method for preparing cross-linked gels of hyaluronic acid and products containing such gels. The cross-linking HA or HA/hydrophilic polymers (polysaccharide or protein) and the divinyl sulfone was carried out at 20° C. in a pH>9 solution. In the 1%-8% dry solids content of mixture, HA contains 5%-95% of dry solids content.

(15 ) Liu et al., 1999, U.S. Pat. No. 5,866,165,states a matrix and a method for preparing it are provided to support the growth of bone or cartilage tissue. A polysaccharide is reacted with an oxidizing agent to open sugar rings on the polysaccharide to form aldehyde groups. The aldehyde groups are reacted to form covalent linkages to collagen. Collagen and polysaccharide used to form matrix are present in a range of 99:1 to 1:99 by weight, respectively. 1% to 50% of the repeat units in polysaccharide are oxidized to contain aldehyde groups.

(16) Pitaru et al., 1999, U.S. Pat. No. 5,955,438,states a method for producing a collagen matrix which may be formed into a membrane useful in guided tissue regeneration. A collagen matrix comprises collagen fibrils which are incubated with pepsin in a solvent, and are then cross-linked to one another by a reducing sugar. Finally, the matrix is subjected to critical point drying.

(17) Pierschbacher et al., 1999, U.S. Pat. No. 5,955,578,states a method for producing polypeptide-polymer conjugates active in wound healing. A synthetic polypeptide comprising the amino acid sequence dArg-Gly-Asp is bonded to a biodegradable polymer via a glutaraldehyde cross-linking agent. The purpose of synthetic matrix is to promote cell attachment and migration.

(18) Hall et al., 1998, U.S. Pat. No. 5,800,811,states a method for producing an artificial skin. An artificial skin is prepared by impregnating a collagen with a transforming growth factor-beta, and incubating the impregnated matrix with a source of stem cells.

(19) Stone et al., 1989, U.S. Pat. No. 5,880,429,states a method for producing a prosthetic meniscus. A pore size in the range 10-50 microns of prosthetic meniscus is formed by type collagen fibrils (65%-98% by dry weight ) and glycosaminoglycan molecular (chondroitin-4-sulfate; chondroitin-6-sulfate; dermatan sulfate or hyaluronic acid; 1%-25% by dry weight ) and which is adapted for in growth of meniscal fibrochondrocytes.

(20 ) Stone, 1992, U.S. Pat. No. 5,108,438,states a method for producing a prosthetic inter-vertebral disc. The disc includes a dry,porous,volume matrix of bio-compatible and bio-resorbable fibers which may be interspersed with glycosaminoglycan molecules (0-25% by dry weight).

The cross-linking agent is selected from the group consisting of glutaraldehyde, carbodiimides and so on.

(21 ) Silver et al., 1987, U.S. Pat. No. 4, 703,108,states a method for preparing biodegradable collagen-based matrix in sponge or sheet form. HA and collagen are added to a dilute HCl solution of pH 3.0 and the mixture is homogenized in a blender. The solution is then poured into a vacuum flask and de-aerated at a vacuum, and then cross-linked with carbodiimide. After then, the matrix is allowed to air dry or freeze dry. The product of collagen-based matrix is cross-linked by immersion in an aqueous solution containing 1% by weight of cyanamide at pH 5.5 for a period of 24hrs at 22 ° C. After removal, the matrix is washed in several changes of water over 24 hrs,frozen and freeze dried at −65° C. in a vacuum.

(22) Silver et al., 1990, U.S. Pat. No. 4, 970,298,states a biodegradable collagen matrix having a pore size and morphology which enhances the healing of a wound. Collagen in HCl at pH from 3.0 to 4.0 is added to the fibronectin in an acid solution pH 3.0 to 4.0 and the mixture is dispersed in a blender. Collagen dispersions to be converted into sponge are frozen at −100° C. before freeze drying at −65° C. The matrix is cross-linked by two cross-linking steps with carbodiimide before dehydrothermal, or cross-linked with carbodiimide after dehydrothermal.

### SUMMARY OF THE INVENTION

To base on the reports of patents and references, the general preparation of the polysaccharide-protein bio-composites is under the acid condition, a polysaccharide-protein fiber precipitate is formed by the ionic bond formation of the mixture of little polysaccharide (less than the 15% weight of collagen) and protein, and then form the covalent bond with the cross-linking reagent, a non-directional fiber sponge or porous matrix is produced after washing, filtration and lyophilization. The defect of this procedure is just can only produce a porous matrix of fiber structure and non-homogeneous composite, it is difficult to form the different types of composites it depends on the need of impalpable matrices. In a general experiment, a piece of precipitate was homogenized by chopping to many small segment, and the homogenized slurry was then poured into the different shaped mold that the experiment wanted, then lyophilized. The developmental techniques of this invention can prepare the different ratio of the mixture solution, the different pH value of the homogeneous polysaccharide-protein solution, and then can be process to different types of the bio-composites (such as membrane, sponge, fiber, tube or micro-granular and so on) After thorough cross-linking reaction with the water/organic solution, a homogeneous, good bio-compatible, biodegraded, prolong enzymatic degradation and fine physics of impalpable bio-composite is formed.

The advantage of this invention is that the polysaccharide-protein solution can be prepared under wide range of pH value, not only in the acid condition, and the weight ratio of polysaccharide to protein is 2/98 to 90/10. In a traditional experiment, the collagen is usually as a major material and the polysaccharide is as an additive, the maximal ratio of polysaccharide to collagen is around 20%. Besides, the matrix solution that produce from this invention is with homogeneous density and porosity, and can be manufactured to various types, including the shape of membrane, sponge, fiber, tube or micro-granular and so on. It can also avoid the loss of polysaccharide and reduce the reaction time to only 2-4hrs while react the cross-linking reaction with carbodiimide in the little acid of organic solution.

In most cases of the previous study, the aldehyde was usually as a cross-linking reagent, but when used the carbodiimide as a reagent, the cross-linking reaction was always finished in the water and the reaction time may need more than 24 hrs.

There are many advantages in this invention and the developmental techniques of this invention are also never have described in previous study. Therefore, the different shapes of the cross-linking polysaccharide-protein bio-composites contain highly commercial apply and are suitable for a wide variety of biomedical, materials engineering, histological engineering, medical equipment, pharmacy and cosmetic uses. Other features and advantages of the invention will be apparent from the following description of the preferred embodiments thereof, and from the claims.

### DESCRIPTION OF THE PREFERRED EMBODIMENTS

This invention relates to a new method for producing different types of cross-linking polysaccharide-protein bio-composites. The characteristic of this method is that the various ratio of the homogeneous the polysaccharide material, such as membrane, sponge, fiber, tube and micro-granular. After cross-linking reaction, a bio-compatible, biodegraded, prolong enzymatic degradation, good mechanical strength, on-toxicity and impalpable bio-composite is formed. It is highly suitable for the application of biomedicine, histological engineering, materials engineering, medical equipment and cosmetic. The use of the bio-composite including as to hemosats, vascular sealants, orthopedic implant coatings, vascular implant coatings, dental implants, wound dressings, anti-adhesion barriers, platelet analyzer reagents, research reagents, engineering of cartilage, artificial tendons, blood vessels, nerve regeneration, cornea implants, cell preservation solutions and growth factor/drug delivery. According to the practice of this invention, the highly additional value of by products can be produced. It is very useful for commercial utilization.

This invention is related with a new method for producing polysaccharide-protein bio-composites, comprising the step of:

(a) preparing a polysaccharide solution.

(b) preparing a protein solution.

(c) adjusting the pH and salt content of the step (a) and (b) to moderate ranges, and then according to the design of experiment, the well mixed solution can be prepared to different shapes, such as membrane, porosity, sponge, tube or micro-granular and so on.

(d) immerging the matrix in the water/organic solution that contains the cross-linked reagent, and reacting under the moderate pH and temperature.

(e) washing the matrix several times selectively, and immersing in the salt solution that is chosen from the group consisting of sodium chloride, dibasic sodium phosphate or the mixture of both.

The matrix was then further washed several times with large volumes of de-ionized water and lyophilized.

As above (a) described, the polysaccharide is chosen from the group consisting of hyaluronic acid, carboxymethyl cellulose, pectin, starch, chondroitin-4-sulfate, chondroitin-6-sulfate, alginate, chitosan, agar, carragenan and guar gum.

As above (b) described, the protein solution is chosen from the group consisting of collagen, gelatin, or the mixture of both.

As above (c) described, the preferred pH value is in a range between 3 and 11,and the change of pH was adjusted with the acetic acid, hydrogen chloride, sodium hydroxide, potassium hydroxide, or the mixture of both that can donate the proton group of acid or the hydroxyl group of alkalinity.

The total dry solids content of polysaccharide-protein mixture solution is in a range between 0.2% and 4.0%, but the weight percent of polysaccharide is in a range between 2% and 98%. The concentration of salt is in a range between 0.05 M and 0.25 M that depends on the selection of the various kinds of acid and hydroxyl compounds.

As above (c) described, the procedures of the different shapes of the material are illustrated as the following:

(1) The film matrix is formed by casting the degas mixture of polysaccharide and protein solution into a mold and allows to dry to yield a film in an oven at 35° C.

(2) The porosity matrix is formed by casting the degas mixture of polysaccharide and protein solution into a mold in a refrigerator at −80° C. and allows to vacuum dry to yield a porous structure in a freeze-dry drying, the porosity of matrix is in the form of a pore morphology with the interconnectivity structure.

(3) The powder matrix is formed by dropping the degas mixture of polysaccharide and protein solution into the freezing solution at −80° C. with a moderate size of the syringe, and allows to vacuum dry to yield a powder matrix in a freeze-dry drying.

(4) The fiber matrix is formed by squeezing the degas mixture of polysaccharide and protein solution into the coagulant of organic solvent with the squeezer apparatus, and allows to dry to yield a fibrous matrix of 50 um-1 mm thickness.

As above described, the coagulant solution comprises water and organic solvent. The organic solvent is chosen from the group consisting of 1,4-dioxane, chloroform, methylenr chloride, N,N-dimethylformamide, N,N-dimethylacetamide, ethyl acetate, acetone, methyl ethyl ketone, methanol, ethanol, propanol, isopropanol, butanol and the mixture of each organic solvent; the weight fraction of organic solvent in the coagulant solution is between 60% and 100%.but the preferred weight fraction of organic solvent is between 75% and 100%, but the preferred weight fraction of organic solvent is between 75% and 100%. The ketones and alcohol can be mixed with any ratio.

As above step (d) described, the preferred cross-linking agent is the carbodiimide, and the carbodiimide is selected from the group consisting of 1-methyl-3-(3-dimethylaminopropyl)- carbodiimide, 3-(3-dimethylaminopropyl)-3-ethyl- carbodiimide, 1-ethyl-3-(3-dimethylaminopropyl)- carbodiimide or the mixture of each group.

As above step (d) described, the water/organic solution contains 5%-50% water of ethanol or acetone solution, but the preferred content is 5%-30%. The pH of mixture is in a range between 4 and 5.5, the temperature of reaction is at 20° C. -45° C. ,the reaction time is 1-6 hrs, but the preferred time is in a range between 2-4 hrs.

As above step (e) described, the immersed concentration of salt is in a range between 0.15-4 M solution that consists of sodium chloride, dibasic sodium phosphate or the mixture of both. The immersed time is in a range between 30 mins and 3 hrs.

The invention is described in more detail in the following example. These examples are giving by way of illustration and are not intended to limit the invention except as set forth in the claims. The preferred for producing different shapes of polysaccharide-protein bio-composites is described in more detail in the following samples.

### Example 1

### The Preparation of Hyaluronic Acid/Collagen Matrix

HA (60 mg) and collagen (40 mg) were dissolved in the different conditions, respectively (as table 1 described),and then the two prepared solutions were mixed together to form a mixture that the weight ratio of HA to collagen is 3 to 2 and the total dry solid content is 1%.

The resulting solution was cast into a mold of Teflon to yield cross-linked film. Sample 1D and 1E had the optimal morphology and physics after cross-linking.

TABLE 1

| Sample | 1A | 1B | 1C | 1D | 1E | 1F | 1G |
|---|---|---|---|---|---|---|---|
| HA^{a} | H₂O | 0.1N | 0.1M | H₂O | H₂O | H₂O | H₂O |
| solvent | | NaCl | CH₃COOH | | | | |
| Collagen | 0.5M | 0.1M | 0.1N | 0.1M | After | adjust pH | 0.5M |
| | CH₃COOH | CH₃COOH | NaCl | CH₃COOH | dissolved in | 7 by HCl | CH₃COOH |
| | | | | | 0.5M acetic | | and 1N |
| | | | | | acid, | | NaOH |
| | | | | | adjust pH by | | mixture |
| | | | | | 1N NaOH | | solution |
| NaCl | — | — | — | 30 mg | — | — | — |
| mixed | white fiber | transparence | transparence | transparence | transparence | ine fiber | white fiber |
| solution | precipitate | low iscosity | | | | precipitate | precipitate |
| 1N | few drops, | — | — | — | — | — | — |
| NaCl | fiber | | | | | | |
| | precipitate | | | | | | |
| | and then | | | | | | |
| | dissolved | | | | | | |
| PH | ˜9 | ˜8 | ˜7 | ˜3 | ˜6 | ˜7 | ˜6 |
| morphology | fine fiber | semi | semi | white density | white density | fine fiber | white |
| | on the | transparence | transparence | | high | on the | |
| | surface | | | | toughness | surface | |

### EXAMPLE 2

### The Preparation of HA/Gelatin Matrix

HA (50 mg) was dissolved in 5 ml of pure water. sodium chloride (30 mg) was gradually added to the solution of gelatin (50 mg) in 5 ml of warm water (more than 55° C.)

The two prepared solution were mixed together to form a 10 ml mixture that the pH of solution is around 6.5, the weight ratio of HA to collagen is 1 to 1 and the total dry solids content is 1%.

The resulting solution was cast into a mold of Teflon plate and allowed to dry under oven to yield a transparent film.

### EXAMPLE 3

### The Preparation of Different Salt Concentration of HA/Collagen Matrix After Neutralization

HA (60 mg) was dissolved in pure water. Collagen (40 mg) was dissolved in 0.5 M acetic acid solution, and then neutralized with sodium hydroxide. Adjust the salt concentration of neutralization and the pH to 6 by changing the volume ratio of water, acetic acid and sodium hydroxide (as table 2 described). The two prepared solution were mixed together to form a 10 ml mixture that the weight ratio of HA to collagen is 3 to 2 and the total dry solids content is 1%.

The resulting solution was cast into a mold of Teflon plate and allowed to dry under oven to yield a film.

TABLE 2

| Sample | 2A | 2B | 2C |
|---|---|---|---|
| H₂O (ml) | 5.5 | 7.0 | 8.5 |
| 0.5M CH₃COOH | 3.0 | 2.0 | 1.0 |
| 1N NaCl | 1.5 | 1.0 | 0.5 |
| Salt conc of | 0.15 | 0.1 | 0.05 |
| neutralization. (M) | | | |

### EXAMPLE 4

### The Preparation of the Different pH of HA/Collagen Matrix

HA (60 mg) was dissolved in pure water. Collagen (40 mg) was dissolved in 0.5 M acetic acid solution, and then neutralized with sodium hydroxide. Adjust the salt concentration of neutralization to 0.15 M and the pH value by acetic acid and sodium hydroxide (as table 3 described). The two prepared solution were mixed together to form a 10 ml mixture that the weight ratio of HA to collagen is 3 to 2 and the total dry solids content is

The resulting solution was cast into a mold of Teflon plate and allowed to dry under oven to yield a transparent film.

TABLE 3

| Sample | 3A | 3B | 3C |
|---|---|---|---|
| H₂O (ml) | 3.5 | 5.5 | 5.44 |
| 0.5 M CH₃COOH | 5.0 | 3.0 | 3.0 |
| 1N NaCl (ml) | 1.5 | 1.5 | 1.56 |
| PH value | 4.7 | 6.0 | 11.0 |

### EXAMPLE 5

### The Preparation of the Different Ratio of HA/Collagen Matrix

HA was dissolved in pure water. Collagen was dissolved in 0.5 M acetic acid solution, and then neutralized with sodium hydroxide. Adjust the salt concentration of neutralization to 0.15 M and the pH to 4.7 by changing the volume ratio of water, acetic acid and sodium hydroxide is 3.5:5:1.5. The two prepared solution were mixed together to form a 10 ml mixture that the weight ratio of HA to collagen is described as table 4 and the total dry solids content is 1%.

The resulting solution was cast into a mold of Teflon plate and allowed to dry under oven to yield a transparent film.

TABLE 4

| Sample | 4A | 4B | 4C | 4D | 4E | 4F |
|---|---|---|---|---|---|---|
| HA (mg) | 90 | 80 | 60 | 50 | 20 | 2 |
| Collagen (mg) | 10 | 20 | 40 | 50 | 80 | 98 |
| Weight ratio | 9:1 | 4:1 | 3:2 | 1:1 | 1:4 | 1:49 |

### EXAMPLE 6

### The Preparation of the Different Total Dry Solids Content of HA/Collagen Matrix

HA was dissolved in pure water. Collagen was dissolved in 0.5 M acetic acid solution, and then neutralized with sodium hydroxide. Adjust the salt concentration of neutralization to 0.15 M and the pH to 4.7 by changing the volume ratio of water, acetic acid and sodium hydroxide is 3.5:5:1.5. The two prepared solution were mixed together to form a 10 ml mixture that the weight ratio of HA to collagen is 3 to 2 and the total dry solids content is described as table 5.

The resulting solution was cast into a mold of Teflon plate and allowed to dry under oven to yield a transparent film.

TABLE 5

| Sample | 5A | 5B | 5C |
|---|---|---|---|
| HA (mg) | 120 | 60 | 30 |
| Collagen (mg) | 80 | 40 | 20 |
| Solid content (%) | 2 | 1 | 0.5 |

### EXAMPLE 7

### The Preparation of the Fiber HA/Collagen Matrix

HA (100 mg) was dissolved in 3.5 ml of pure water. Collagen (100 mg) was dissolved in 5 ml of 0.5 M acetic acid solution, and then neutralized with 1.5 ml of 1 N sodium hydroxide. The salt concentration of neutralization is 0.15 M. The two prepared solutions were mixed together to form a mixture that the pH of solution is around 4.7,the weight ratio of HA to collagen is 1 to 1 and the total dry solids content is 2%.

The resulting solution was continual pressed into the 95% alcohol solvent to form a mono-filament fiber by using the different size of syringe, and allowed to dry under oven to yield a HA-protein matrix.

### EXAMPLE 8

### The Preparation of the Micro-Granular HA/Collagen Matrix

HA (100 mg ) was dissolved in 3.5 ml of pure water. Collagen (100 mg) was dissolved in 5 ml of 0.5 M acetic acid solution, and then neutralized with 1.5 ml of 1 N sodium hydroxide. The salt concentration of neutralization is 0.15 M. The two prepared solutions were mixed together to form a mixture that the pH of solution is around 4.7,the weight ratio of HA to collagen is 1 to 1 and the total dry solids content is 2%.

The micro-granular matrix was formed by dropping the resulting solution into the liquid Nitrogen and lyophilized.

### EXAMPLE 9

### The Preparation of the Porous HA/Collagen Matrix

HA (100 mg) was dissolved in 3.5 ml of pure water. Collagen (100 mg) was dissolved in 5 ml of 0.5 M acetic acid solution, and then neutralized with 1.5 ml of 1 N sodium hydroxide. The salt concentration of neutralization is 0.15 M. The two prepared solutions were mixed together to form a mixture that the pH of solution is around 4.7,the weight ratio of HA to collagen is 1 to 1 and the total dry solids content is 2%.

The resulting solution was cast into a mold of Teflon plate at −80° C. and allowed to dry to yield a porous sponge matrix after lyophilization.

### EXAMPLE 10

### The Effect of Cross-Linked Agent on the Chemical Cross-Linking of HA/Collagen

The film of sample 5A was chopped to equal pieces and immersed in the EDC for 2 hrs at 30° C. (experimental conditions were as table 6 described). The matrix was then washed 3 times with 80% acetone solution, each time is 20 mins. After then, the matrix was further washed 3 times with de-ionized water, each time is also 20 mins. Finally, the matrix was spread and dried. The cross-linked film was immersed in 0.15 M sodium chloride solution for swelling test, incubated 5 days with gentle shaking at 37° C. ,then the swelling behavior was observed. Table 6 results showed that in order to avoid the dissolution of matrix and enhance the cross-linking efficiency, the cross-linking of matrix was only carried out in the water/organic solution that contained the cross-linked agent (sample 6D,6E)

TABLE 6

| Sample | 6A | 6B | 6C | 6D | 6E |
|---|---|---|---|---|---|
| EDC conc. | 2.3 | 2.3 | 2.3 | 2.3 | 2.3 |
| (wt %) | | | | | |
| Solvent | H₂O | PH4.7 | PH4.8 | 80% | 80% |
| | | solution | solution | ethanol | acetone |
| Morphology | thinness | thinness | thinness | normal | normal |
| Dissolve test | soluble | soluble | soluble | insoluble | insoluble |

### EXAMPLE 11

### The Effect of Concentration of Cross-Linked Agent on the Chemical Cross-Linking of HA/Collagen

The film of sample 5A was chopped to equal pieces and immersed in 80% acetone solution at pH 4.7,that contained EDC for 2hrs at 30° C. (experimental conditions were as table 7 described). The matrix was then washed 3 times with 80% acetone solution, each time is 20 mins. After then, the matrix was further washed 3 times with de-ionized water, each time is also 20 mins. Finally, the matrix was spread and dried. The cross-linked film was immersed in 0.15 M sodium chloride solution for swelling test, incubated 5 days with gentle shaking at 37° C., then the swelling behavior was observed. Hyaluronidase (220 U/ml) was dissolved in 0.15 M sodium chloride. Film was weight and placed in the enzyme solution for the test of enzyme degradation. The solution was taken out after 24 hrs for uronic acid assay, and then the percent of hydrolysis of HA film was calculated. Table 7 results showed that the rate of enzyme degradation of the cross-linked film which procedure by this method was reducible significantly.

TABLE 7

| Sample | 7A | 7B | 7C | 7D | Control |
|---|---|---|---|---|---|
| EDC (wt %) | 0.625 | 1.25 | 2.5 | 5 | — |
| Dissolve test | insoluble | insoluble | insoluble | insoluble | soluble |
| HA enzyme | 1.87 | 1.5 | 0.68 | 1.02 | 31.13 |
| degradation | | | | | |
| (%) | | | | | |

### EXAMPLE 12

### The Chemical Cross-Linking Reaction of the Porous HA/Collagen Matrix1

The porous sponge of sample 9 was placed in an oven and vacuumed for 3 hrs at 110° C. Dried specimens was then immersed in the 80% acetone solution for 30 mins, and then transferred to 80% acetone solution at pH 4.7 that contained 2.5% EDC.

The specimens was taken out after 2 hrs reaction at 30° C., and then washed 3 times with 80% acetone, each time is 20 mins. After then, the specimens was further immersed in IM sodium chloride for 20 mins, and washed 3 times with deinoized water, each time is also 20 mins. Finally, the specimens was spread and dried.

### EXAMPLE 13

### The Cross-Linking HA/Collagen for the Cell Growth and Cyto-Toxicity

The films sample 4C, 4D and 4E were immersed in the 80% acetone solution at pH 4.7 that contained 2.5% EDC. The film was taken out after 2 hrs reaction at 30° C. ,and then washed 3 times with 80% acetone, each time is 20 mins. After then, the film was further immersed in 1 M sodium chloride for 20 mins, and washed 3 times with de-ionized water, each time is also 20 mins. Finally, the film was spread and dried.

The cross-linked film was placed in the cells of cell culture plate. Immortalized mouse 3T3 fibroblast cell and human fibroblast cell were seeded on the film matrix for the observation of growth (table 8,9). The results of cell seeding experiment showed that cell can growth well on the film matrix, and all the cells were alive while stained with neutral red dye. It showed that the film matrix was noncyto-toxicity for the human and mouse cell growth.

Although the invention has been described with reference to the presently preferred embodiments, it should be understood that various modifications can be made by those skilled in the art without departing from the invention. Accordingly is set out in the following claims.

TABLE 8

| | Seeding of | | | |
|---|---|---|---|---|
| | cell No | 1st day | 2nd day | Third day |
| Sample | (×10⁴ cell/ml) | (×10⁴ cell/ml) | (×10⁴ cell/ml) | (×10⁴ cell/ml) |
| Cross-linked 4C | 4 | 1.8 | 2.4 | 4.8 |
| Cross-linked 4D | 4 | 2.4 | 4.2 | 7.4 |
| Cross-linked 4E | 4 | 1.4 | 1.8 | 3.4 |

TABLE 9

| | Seeding of cell | 1st day | 2nd day | Third day |
|---|---|---|---|---|
| Sample | No (×10⁴ cell/ml) | (×10⁴ cell/ml) | (×10⁴ cell/ml) | (×10⁴ cell/ml) |
| Cross-linked 4C | 4 | 1.2 | 2.2 | 5.0 |
| Cross-linked 4D | 4 | 2.6 | 4.4 | 7.4 |
| Cross-linked 4E | 4 | 1.6 | 2.4 | 4.0 |

1. A method for producing cross-linked polysaccharide-protein bio-composites, comprising the type of:
(a) preparing a mixture of the polysaccharide solution and protein solution, the weight ratio of polysaccharide and protein is in a range of 20/80 to 80/20.
(b) adjusting the pH value between 3 and 11 by acid and hydroxyl compound.
(c) processing the cross-linking reaction in the water/organic solution that contains the cross-linked reagent.

2. The method of claim 1, wherein said step (a) the polysaccharide is chosen from the group consisting of hyaluronic acid, carboxymethyl cellulose, pectin, starch, chondroitin-4-sulfate, chondroitin-6-sulfate, alginate, chitosan, agar, carragenan and guar gum.

3. The method claim 1, wherein said step (a) the protein solution is chosen from the group consisting of collagen, gelatin, or the mixture of both.

4. The method claim 1, wherein said step (b) the acid compound chosen from the group consisting of acetic acid, hydrogen chloride, or the mixture of acetic acid and hydrogen chloride.

5. The method claim 1, wherein said step (b) the hydroxyl compound is the hydroxyl group donor of alkalinity, the optimal choice is from the group consisting of sodium hydroxide, potassium hydroxide, or the mixture of both.

6. The method claim 1, wherein said the protein solution is that collagen is dissolved at pH 3 solution, while the solution is made of polysaccharide solution.

7. The method claim 1, wherein said the protein solution is that collagen is dissolved in alkaline solution, while the polysaccharide is dissolved in acid solution, the pH range is between 5 to 11 after mixing the both solutions.

8. The method claim 1, wherein said the protein solution is that collagen is dissolved in acid solution, while the polysaccharide is dissolved in alkaline solution, the pH range is between 5 to 11 after mixing the both solutions.

9. The method claim 1, wherein said the protein solution is that gelatin is dissolved in de-ionized water, and the ion strength is adjusted by sodium chloride.

10. The method claim 1, wherein said step (b) the mixture solution is adjusted by acid and hydroxyl compound, and allow to spread to yield a film after degas, or cast into a mold and freeze-dry to yield a proposity matrix, or emulsify to yield a powder matrix, or produce a fiber matrix by using the squeezer equipment.

11. The method claim 10, the film matrix is formed by casting the degassed mixture of polysaccharide and protein solution into a mold and allows drying under oven at 20° C. to 45° C. to yield a film.

12. The method claim 10, the porosity matrix is formed by casting the degassed mixture of polysaccharide and protein solution into a mold in a refrigerator at −30° C. to −100° C. and allows to vacuum dry under freeze-dry drying to yield a porous structure, the porosity of matrix is in the form of a pore morphology with the interconnectivity structure.

13. The method claim 10, the powder matrix is formed by dropping the degassed mixture of polysaccharide and protein solution into the freezing solution at −30° C. to −1 00C with a moderate size of the syringe, and allows to vacuum dry under freeze-dry drying to yield a powder matrix.

14. The method claim 10, the fiber matrix is formed by squeezing the degassed mixture of polysaccharide and protein solution into the coagulant of organic solvent with the squeezer apparatus, and allows drying to yield a fibrous HA bio-composite of 50um-1 mm thickness.

15. The method claim 14, wherein said coagulant solution comprises water and organic solvent; the organic solvent is chosen from the group consisting of 1,4-dioxane, chloroform, methylene chloride, N, N-dimethylformamide, N, N-dimethylacetamide, ethyl acetate, acetone, methyl ethyl ketone, methanol, ethanol, propanol, isopropanol, butanol and the mixture of each organic solvent ; the weight fraction of organic solvent in the coagulant solution is in a range between 60% and 100%.

16. The method claim 15, wherein the ketones and alcohol can be mixed with any ratio, and the preferred weight fraction of organic solvent is in a range between 75% and 100%.

17. The method claim 1, wherein said step (c) the cross-linking agent is a carbodiimide.

18. The method claim 17, wherein said the carbodiimide is selected from the group consisting of 1-methyl-3-(3-dimethylaminopropyl)-carbodiimide, 3-(3-dimethylaminopropyl)-3-ethyl-carbodiimide, 1-ethyl-3-(3-dimethylaminopropyl)-carbodiimide and the mixture of each group.

19. The method claim 1, wherein said step (c) the water/organic solution contains 5%-50% water of ethanol or acetone solution; adjust the pH of mixture solution to 4-5.5,the weight percent of carbodiimide is 0.5%-25%; the temperature of reaction is 20° C-45° C., the reaction time is 1-6hrs.

20. The method claim 19, wherein said the water/organic solution contains 5%-30% water of ethanol or acetone solution; adjust the pH of mixture solution to 4-5.5,the weight percent of carbodiimide is 1%-5% ; the reaction time is 2-4hrs.

21. The method claim 1, wherein said step (c) the cross-linked of water-insoluble polysaccharides can be washed with water/organic solution, and washing with distilled water after immersing in the salt solution.

22. The method claim 21, wherein said the water/organic solution contains 5%-50% water of ethanol or acetone solution, the immersion time is 0.5-3hrs.

23. The method claim 22, wherein said the water/organic solution contains 5%-30% water of ethanol or acetone solution.

24. The method claim 21, wherein said the salt solution is chosen from the group consisting of sodium chloride, dibasic sodium phosphate and the mixture of both, the concentration of salt is 0.15-4 l M.
